(19) United States
(12) Patent Application Publication
(10) Pub. No.: US 2009/0143653 A1
(43) Pub. Date: Jun. 4, 2009

(54) MINIATURE DEVICE FOR ANALYZING PHYSICOCHEMICAL PROPERTIES OF THE SKIN

(75) Inventors: Paul Laurens, Pommier De Beaurepaire (FR); Pascal Ransch, Meylan (FR)

Correspondence Address: HESLIN ROTHENBERG FARLEY & MESITI PC, 5 COLUMBIA CIRCLE, ALBANY, NY, 12203, US

(73) Assignee: INTUISKIN, Crolles (FR)

(21) Appl. No.: 11/574,492

(22) PCT Filed: Sep. 2, 2005
(86) PCT No.: PCT/FR2005/050703

§ 371 Date: Feb. 28, 2007

(30) Foreign Application Priority Data
Sep. 3, 2004 (FR) 0451969

(51) Int. Cl. A61B 5/00

(52) U.S. Cl. 600/306

(57) ABSTRACT

A chip card for analyzing physicochemical properties of a cutaneous surface includes at least one body sensor which can deliver a signal representing a measurement of a physicochemical property of the cutaneous surface, and a processing unit used to treat signals delivered by the body sensor and an optional environment sensor and to then store signals in a storage unit on the card. An electrical energy source on the card provides electrical power to the body sensor and the processing unit.

### TECHNICAL FIELD

The invention relates to the field of cosmetology and dermatology. It relates more particularly to a portable and self-contained miniature apparatus for analyzing at least one physicochemical parameter of a cutaneous surface, for diagnosing a potential symptom, and if necessary, indicating the most appropriate products for the treatment and/or treatments thereof.

Furthermore, such an apparatus may also take a measurement of a parameter of the external environment in which the measurement of the physicochemical property is taken.

### PRIOR ART

In general, apparatus for taking measurements of a physicochemical property of a cutaneous surface comprise both a computerized processing unit and a set of sensors positioned in a probe which contacts the cutaneous surface.

Such an apparatus has been described in particular by the applicant in document WO 03/037184. This apparatus is designed for being installed in a dermatological centre or any other firm for dermatological analyses. It is in the form of a computer equipped with acquisition cards and a probe. It is therefore relatively bulky and therefore difficult to transport. In consequence, the number of potential users of this type of apparatus is limited, or the interested parties are obliged to travel to a place equipped with such an apparatus.

Chip cards are also known as described in document US-2002/0095587, particularly for payment, equipped with a biometric sensor for permitting the use of the card, after comparing the biometric measurement taken with previously stored data. Such a biometric sensor can be used to identify a finger or retinal imprint, to analyze the chemistry of the skin, or to take other measurements of a physicochemical parameter specific to an individual, suitable for formally determining the identity of a person.

In this case, however, such a chip card cannot provide the user with data on a physicochemical parameter of the cutaneous surface. The data delivered by the sensor are not stored in the card, because the said card must be introduced into a reader when identification is necessary. The card reader is therefore used to transfer immediately the data delivered by the biometric sensor to a system which compares the data measured with similar data stored in a card or a computerized system.

Contrary to this chip card, it is the object of the invention to permit the analysis and display of data that is a function of at least one physicochemical parameter of a cutaneous surface. This measurement can be taken regardless of the geographic location and at any time whatsoever, using a miniature and self-contained apparatus.

A further object of the invention is to permit the determination of at least one parameter of the external environment in which the measurement of the physicochemical property is taken or even when no measurement of a physicochemical property is taken.

Hence the object of the invention is not to perform transdermal injections of active substance, as described in document DE-115930. This document describes a patch equipped with communication means for transmitting data to a processing unit. In this case, however, the data transmitted concern the effectiveness of injection of the active substance. In fact, the processing unit is informed by the patch when the substance is no longer present therein, or when the patch is not correctly positioned on the surface of the skin. Thus, such a patch cannot perform an analysis of the physicochemical properties of the skin.

### SUMMARY OF THE INVENTION

The invention therefore relates to an analytical device in the form of a chip card comprising:
at least one body sensor which can deliver a signal representing a measurement of a physicochemical property of a cutaneous surface;
a processing unit used to treat the signal delivered by the body sensor and then to store the said signal in a storage unit;
an electric power source for supplying the body sensor and the processing unit.

In other words, at least one body sensor can supply data that is a function of a physicochemical property of the cutaneous surface of a user. The signal(s) delivered by the sensor(s) are received by a processing unit incorporated within the card, and are then processed so as to permit their storage in the storage unit. The processing unit and the sensors are supplied with electric power to deliver a signal. Thus, an electric power source is integrated within the chip card.

In practice, the analytical device may comprise at least one environment sensor which can deliver a signal representing a measurement of a parameter of the external environment.

In other words, at least one environment sensor is used to supply data that is a function of a parameter of the external environment, such as the temperature, humidity, pressure or radiation of fixed or variable frequency electromagnetic waves such as ultraviolet, infrared, microwave or sound waves.

According to a particular use of the device which comprises both types of sensors simultaneously, the body and environment measurements can be taken simultaneously.

According to another method of use of the device, the measurements can be taken independently at different times according to the user's needs. And, for certain applications, it is also possible to use one or the other of the two types of sensors in order to take measurements of one and the same parameter. In fact, in certain particular applications, the user may also only take a measurement of one parameter of the external environment, such as ultraviolet radiation, without necessarily measuring a physicochemical property of a cutaneous surface.

Advantageously, the device may comprise a microcamera. This camera may particularly incorporate a lens or a light source for evaluating the dimensional or colorimetric characteristics of the skin. The microcamera may be monochrome or colour according to the needs of the measurement to be taken.

In fact, such a microcamera can be used to measure the length of a wrinkle, its depth, or the diameter of a pigment spot forming a mole.

According to a particular embodiment, one or the other of the body or environment sensors may be formed by such a microcamera.

To improve the taking and subsequent use of the image, an additional element may be associated with the microcamera, and particularly a lighting device, a wide angle lens, a zoom or a specific wavelength filter.

According to a first alternative, the lighting can be provided by a light source inside the device.

According to a second alternative, the lighting may also be provided using an external light source.

Furthermore, regardless of the alternative used, the light source may comprise light emitting diodes (LED) or organic light emitting diodes (OLED) emitting in the same specific wavelength or a plurality of LED or OLED emitting in different wavelengths. These LED or OLED may be arranged in circular, triangular or rectangular arrangements around the camera.

The monochrome or multicolour lighting modes can be selected by the apparatus automatically or by the user via one or more man-machine interfaces.

In a particular embodiment, a positioning aid can be integrated and provided by means of at least one oriented LED or OLED pointer. A pattern comprising a simple or complex geometry is projected on the zone to be measured. The image reflected by this pattern can then be interpreted manually or automatically by the focusing device thereby adjusting the camera settings.

In the case in which a zoom is incorporated, the system may have means for adjusting the said zoom.

The camera is connected to an electronic unit in charge of managing the setting parameters which are the shutter speed, aperture, or gain. The light and sharpness measurements for the setting are made either through the camera lens or using an additional sensor. The image captured by pressing a control knob can be stored in the incorporated storage unit. The format of the captured image is preferably of the compressed type and compatible with the standard formats in force.

The system may integrate image processing algorithms. A man-machine interface, comprising at least one selection knob, enables the user to make a selection from one or more algorithms available according to the measurement which he wishes to take and particularly:
the length of the lines, small wrinkles or developed microstructures;
the area of the wrinkled zones;
the local or average roughness;
the isotropy of the skin;
the local or average colorimetry;
the area of the hyperpigmented zone;
the measurement of the lipids content;
the concentration of hairs on the skin.

According to a first alternative, the image or the calculation result may be displayed on an incorporated display.

According to a second alternative, the image is only stored in the memory of the system. The image may then be visualized once it is transferred to an apparatus of the computer, television, mobile telephone or pocket organizer type.

In a particular embodiment, the body and environment sensors may be active sensors. In this case, they deliver a signal representing a physicochemical parameter of a cutaneous surface and/or a parameter of the environmental conditions only when they are supplied with electricity. In other alternatives, the sensor(s) may be of the passive type.

In practice, the chip card may comprise man-machine interfaces for performing numerous actions according to the version of the card energized, the selection of the type of skin, and the selection of the algorithm.

The energy source may be in the form of a cell or a battery which, when a measurement is needed of a physicochemical property and/or a parameter of the external environment, supplies the corresponding sensor and the processing unit. When the card is not used, a switch or an electric power management device serves to open the circuit to preserve the electric power stored in the battery and prevent its discharge.

In a first alternative, the electric power source may be a photovoltaic cell. This cell serves to supply the sensor and the processing unit with electric power. In this case, the energy is therefore supplied by the ambient light.

In a second alternative, the electric power source may be a microgenerator converting an external energy source into electrical energy by induction, Hall effect, magneto-resistive, piezoelectric, thermal or mechanical effect.

In order to optimize the electric power consumption, the card may incorporate an energy management system selectively activating and deactivating the electrical members according to preprogrammed modes, thereby managing various power modes. This subsystem may incorporate battery charging modes when the card is inserted into a specific charger.

According to the measurement taken of the physicochemical property, the sensor may be selected particularly from the group comprising:
pH sensors;
cutaneous imprint sensors, suitable for measuring the topography of the cutaneous surface to be analyzed;
skin moisture sensors;
skin temperature sensors;
lipids content sensors;
elastic deformation sensors of the cutaneous surface to be analyzed.

Obviously, for certain applications, the card may comprise a plurality of sensors selected from this group and suitable for measuring different physicochemical properties.

The measurement of the skin pH serves to distinguish a high pH, of about 5.5, and a more acidic pH, close to 5.

The moisture sensor serves to accurately measure the parameter generally qualified as Trans Epidermal Water Loss, abbreviated TEWL.

This parameter corresponds to the evaluation of a mechanism independent of transpiration, reflected by the evaporation of moisture from the underlying layers of the epidermis. This measurement serves, for example, to monitor the hydrolipid film that plays the role of a barrier function for the skin and to define the scale of cutaneous dryness of the skin.

The cutaneous imprint sensor serves to prepare a measurement of the various irregularities of the surface of the skin. This measurement can be taken according to various principles, such as a capacitive, piezo-resistive, piezoelectric, optical or electromagnetic measurement. The determination of the topography of the zone to be analyzed serves to measure the deformity of the skin, the number of wrinkles, their length, area and average depth. The total area of the wrinkles can be determined by calculating the area occupied by the average and deep wrinkles, corresponding respectively to the wrinkles whereof the depth is between 150 and 200 microns, and is greater than 200 microns.

It is also possible to determine the intensity of the main furrows, in order to determine the length of the deepest wrinkles. The determination of the volume of the main wrinkles serves to measure the change of these wrinkles over time.

The measurement of the skin roughness is also an important parameter, because it serves to approach the overall concept of planeity of the skin by characterizing it by an average amplitude value which is the resultant of the various relief irregularities compared to a flat surface. The measurement of this roughness parameter, and of its variation over time, serves to identify the smoothing of the skin resulting from a particular treatment.

The measurement of the temperature or ambient humidity may help to correct certain specific measurements, particularly that of the skin moisture content, that is, the trans epidermal water loss. It also helps to analyze a diagnosis with regard to the atmospheric conditions.

The lipids content sensor serves to determine the status of the cutaneous lipids, particularly for dry skins. This measurement serves to distinguish the mechanisms of cutaneous dryness from the mechanisms of excessive sebum production.

The elastic deformation sensor of the cutaneous surface to be analyzed serves to measure the firmness and elasticity of the skin. This deformation sensor functions on the principle of the application of a negative pressure to a zone of the skin, during a constant time interval. Several successive suctions may be performed in order to measure the skin penetration depth in a part of the card. More precisely, this measurement can be taken via optical sensors or using strain gauges, for example.

The analysis of the various measurements obtained serves to distinguish the instantaneous deformations corresponding to an elasticity mechanism, and the delayed deformations, which can be treated as a viscosity mechanism.

The measurement of the environmental conditions during or between two measurements of the skin provides data considerably improving the interpretation of the results. In fact, since the skin is a living organ having a function of regulating exchanges between the human body and its surroundings, it adapts and modifies its characteristics constantly according to the environmental conditions. The measurement of these conditions may sometimes even be indispensable to accurately evaluate the effectiveness of a cosmetic or medical treatment.

In other cases, it is advantageous to quantify the exposure of the skin to the environmental conditions for an early warning of potential pathological risks. For example, controlled environments such as cold rooms, white rooms, blast furnaces, aircraft cockpits are particularly harmful to the skin, in the same way as the sunlight absorbed when sunbathing or a mountain walk.

Advantageously, the sensor can be made using microsystems. An alternative of active or passive sensors can function according to the principle of MEMS or “Microelectromechanical Systems Technologies”. These sensors are therefore prepared according to technologies using semiconductor, insulating or metallic materials, and chemical etching methods employed in the field of microelectronics. The use of MEMS type sensors serves to concentrate a large number of sensors on a particularly restricted zone, installed on the chip card. This serves to obtain representative results of a localized, homogeneous and characteristic zone.

Due to its very small size, the use of MEMS advantageously serves to decrease consumption and hence increase the service life of the device.

In order to optimize the contact between the skin and the body sensor and hence the measurement efficiency, a device for monitoring the contact pressure, positioning or orientation can be integrated in the card. The operating principle of this monitoring device may be based on the specific properties of the materials used to make the apparatus and/or the mechanical properties according to the deformations imposed by specific cutout geometries and/or sandwich arrangements between the various layers of material.

According to a particular embodiment, active electrical subunits such as piezoelectric actuators and/or MEMS can perform the measurement monitoring function previously described. This monitoring device may be very useful for repositioning the card on a part of the skin undergoing treatment.

Thus the MEMS can simultaneously integrate functions for measuring and monitoring its efficiency by combining electrical and mechanical devices at a very small scale and with very low consumption, meeting the major requirements of the card.

In practice, the card can integrate an active or passive calibration device as required. Calibration serves to correct the potential drifts of the sensors or to adjust the operating point of the system according to the applications.

Depending on the type of measurement to be taken, the data recorded by the sensor may either be displayed directly on the card, or be transferred to a computerized system, or both.

Thus, the card may comprise means for displaying data generated by the processing unit and stored in the storage unit during its display.

In other words, when the measurement is taken, the processing unit transmits data representing the measurement taken to the display means. This data is stored during its display in the storage unit.

In practice, the display means may comprise liquid crystals or light emitting diodes of the LED or OLED type. They can thus serve to quantitatively display a value level according to the measurement taken.

The card can also serve to transfer data delivered by the sensor to a computerized system. In this case, the storage unit may be of the permanent storage type to store data representing the said measurement and for its subsequent transfer. The storage unit may thus comprise a capacitance to store a significant number of measurements without the need to transfer the data contained in the storage unit. Once the measurements are taken, the data can therefore be transferred to a computerized system for analysis. This storage capacity can also be used to store data such as identification codes, as well as calibration data, on the status of the sensor and of the card. It is therefore possible thereby to compile a record of the measurements, and also to calculate a relative variation from one measurement to another.

Advantageously, the computerized system can be associated with a database of treatments and treatment products, serving to advise the user of a treatment and/or the most appropriate product for the state of his skin, this product being selected from a range pre-recorded in the database.

According to a particular embodiment, an expert system can be loaded in the storage unit of the device during its manufacture. This expert system comprises transfer functions having as input parameters the measurements of the sensors and/or the result of the image processing. After calculation, the card displays at least one recommendation which can be selected and validated by the user.

In practice, the device may require the user to select his profile such as his skin type, age, sex, or the place of measurement and this selection can be made during the first use, before a measurement or before the interpretation of the measurement by the expert system. These data are stored in the memory registers and can be called as criteria for selecting the transfer function to be used for processing the measurements or images to follow.

The card is designed to comprise on its surface an interface for transferring the data representing the said measurement when the said card is inserted into a card reader, connected to a computerized system.

In other words, the electrical conduction tracks may be apparent in a localized zone of the chip card in order to authorize the transfer of the data contained in the storage unit to the computerized system. The transfer source is made possible thanks to the use of a card reader linked to the computerized system.

In another alternative, the card may comprise a module for data transmission by radiofrequency. The protocol used to transmit the data may then be in a number of variants and particularly of the Bluetooth or Wifi type.

According to a first alternative, the computerized system may be the PC of the card user. In this case, the measurement can be taken at home, but also in a public place. The miniaturization of the electronic components means that the expression computerized system implies any system, no matter how small it may be, but capable of storing the data in a permanent type of memory and of communicating with a web server. A mobile telephone or portable organizer can thus be used as a computerized system.

According to a second alternative, the computerized system may be a terminal installed in a cosmetic shop. The chip card can particularly be the user's loyalty card. In this case, the card can be used just before the purchase of a product. In this way, the user can identify the product thus appropriate to the state of his skin precisely at the time of purchase of the product.

The card reader may optionally integrate the charger of the electric power source.

The chip card may in particular be supplied with a cosmetic product and thereby permit measurement of the physicochemical property, the measurement of the dimensional and calorimetric characteristics corresponding to the cosmetic product. This chip card can also help to monitor the measurement conditions and the precise tracking of the context of exposure of the skin. The card can thus be directly bonded to the product or inserted into its packaging.

Due to its geometry and its relatively large plane area, the card proposes locations on which locations of an explanatory, educational or advertising nature provide data complementary to the results of the measurements taken.

The system is in the form of several micromodules, each performing one or more functions integrated in one or more generic card platforms. The cards are personalized during assembly.

Thanks to this modular system arrangement and virtually two-dimensional geometry (thickness to width ratio higher than 75), a large number of cards and versions can be made on automated production lines providing the benefit of a very low production cost never yet achieved for such a complete analytical system.

### BRIEF DESCRIPTION OF THE FIGURES

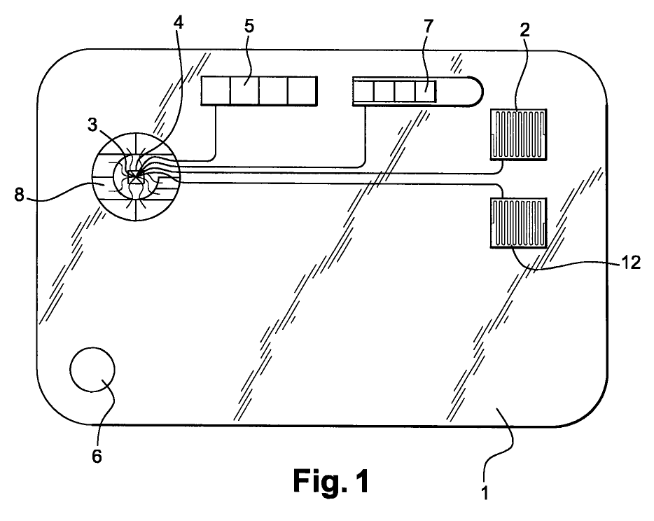

The method for carrying out the invention and the advantages derived therefrom will clearly appear from the description of the embodiment that follows, provided for information and non-limiting, in support of FIG. 1 schematically showing the chip card of the invention.

### MANNER OF DESCRIBING THE INVENTION

As already stated, the invention relates to an analytical device in the form of a chip card equipped with various components for taking a measurement of a physicochemical property of a cutaneous surface.

As shown in FIG. 1, the chip card (1) shows at one of its sides a body sensor (2) for delivering a signal representing the measurement of a physicochemical property.

Once the signal is generated by the body sensor (2), it is transmitted to a processing unit (3) for its storage in a storage unit (4). As shown, the processing unit (3) and the storage unit (4) are contained within the chip positioned in the internal volume of the card (1).

The storage unit (4) can be used to store the data in various ways.

Furthermore the chip card (1) may also comprise an environment sensor (12) which can deliver a signal representing a measurement of a parameter of the external environment. The signal thus delivered by the environment sensor (12) is then sent to the processing unit (3) for its storage in the storage unit (4).

The card may comprise display means (7) enabling the user to directly view data related to the measurement to be taken. In this case, the storage unit (4) serves to store the data during its display.

As shown, the display means (7) use the liquid crystal technology in order to display a value level representing the measurement.

In certain cases, the data may not be displayed directly on the card (1). In this case, the storage unit (4) can be used to store the data permanently. An electric power source (5), shown here in the form of a photovoltaic cell, can supply electric power both to the processing unit (3) and the body sensor (2) when the latter is active. In fact, in this case, to permit the measurement of a physicochemical property, it is necessary to supply the body sensor (2) with electrical energy. A switch (6) serves to cut off this power supply when the measurement is completed. Conversely, when a measurement needs to be taken by the body sensor (2), a simple pressing of the switch (6) serves to energize the body sensor (2).

When the data stored in the storage unit (4) must be transferred to a computerized system, the card (1) is inserted into a card reader connected to the computerized system. An interface (8), flush with one of the sides of the chip card (1), then serves to transfer the data to the memory of the computerized system.

According to a first embodiment, these data can then be analyzed directly by the computerized system to perform an analysis and particularly to compare the measurement with the databases in order to prescribe a product appropriate to the cutaneous surface.

According to a second embodiment, the computerized system can serve as a bridge to a central server suitable for retrieving the data from each chip card. A measurement database is then implemented by the measurements of each of the users.

In consequence, a user equipped with a computer and an internet connection can determine, at anytime and anywhere, the record of the state of his skin and visualize this record particularly by curves.

Depending on the data received by the server, the use of an appropriate product or treatment may be proposed to the user. He may then decide to order the product in question directly and receive it at home without needing to go to a place of sale.

Initially, the determination of the ideal cosmetic product for the physicochemical properties of the user's skin is made by comparing these results with diagnostic criteria prepared, for example, by a scientific team. These diagnostic criteria are themselves part of a diagnostic database, which can be updated and enriched by the statistical analysis of the previous measurements, but also by the analyses of dermatologists equipped with analytical apparatus (for example according to patent WO 03/037184) connected to the central server.

It appears from the above that a chip card according to the invention has many advantages, and particularly:
it serves to make the analysis of a physicochemical property of a cutaneous surface financially affordable to a large number of persons;
it is very compact, and can be taken anywhere, like a credit card;
it can be used to buy a cosmetic product in a shop or to apply it, according to the real need of the skin at the time when the measurement is taken.

1. Chip card for analysing physicochemical properties of a cutaneous surface comprising:
at least one body sensor which can deliver a signal representing a measurement of a physicochemical property of a cutaneous surface;
a processing unit used to treat the signal delivered by the body sensor and then to store the signal in a storage unit; and
an electric power source for supplying electric power to the body sensor and the processing unit.

2. Chip card according to claim 1, further comprising at least one environment sensor which can deliver a signal representing a measurement of a parameter of external environment.

3. Chip card according to claim 1, wherein the chip card comprises a microcamera.

4. Chip card according to claim 2, wherein the at least one body sensor and the at least one environment sensor comprise active sensors.

5. Chip card according to claim 1, wherein the electric power source comprises a photovoltaic cell.

6. Chip card according to claim 1, wherein the at least one body sensor is selected from the group consisting of
pH sensors;
cutaneous imprint sensors, suitable for measuring topography of the cutaneous surface to be analyzed;
skin moisture sensors;
skin temperature sensors;
lipids content sensors;
elastic deformation sensors of the cutaneous surface to be analyzed.

7. Chip card according to claim 4, wherein the at least one body sensor and the at least one environment sensor comprise microelectromechanical systems (MEMS).

8. Chip card according to claim 1, further comprising means for displaying data prepared by the processing unit and stored in the storage unit during display.

9. Chip card according to claim 8, wherein the display means comprises a liquid crystal display or a light emitting diode display.

10. Chip card according to claim 1, wherein the storage unit is of the permanent type for storing data representing measurement and for subsequent transfer of said data to a computerized system.

11. Chip card according to claim 10, further comprising an interface on a surface of the card for transferring the data representing said measurements when said card is inserted into a card reader connected to a computerized system.
